(19) United States
(12) Patent Application Publication
(10) Pub. No.: US 2004/0044349 A1
(43) Pub. Date: Mar. 4, 2004

(54) Anastomosis device delivery systems

(76) Inventors: Gitanjali V. Barry, Fremont, CA (US); Stanley R. Conston, San Carlos, CA (US)

Correspondence Address: VasConnect Inc, Suite 310, 200 Middlefield Road, Menlo Park, CA, 94025, US

(21) Appl. No.: 10/236,060

(22) Filed: Sep. 4, 2002

(51) Int. Cl. A61F 2/06

(52) U.S. Cl. 606/108; 606/155

(57) ABSTRACT

Devices and associated methods for implanting or delivering devices within vessels, lumens, ducts or other tubular organ or graft conduits rapidly, safely and in a minimally invasive manner are described. The subject devices variously include a split-end confinement member adapted to receive an anastomosis device and a plunger to urge a seal of the connector out of a lower opening in the confinement member. Each of the connector and confinement member are preferably surrounded by a reinforcement member to aid in delivery. Such a reinforcement member and the confinement member preferably include an upper opening to receive a tubular flow channel portion or connector lumen of the anastomosis device. In addition, each preferably includes and tip to facilitate vessel entry. The subject methods involve delivery of an anastomotic connector using the subject devices. The methods may be employed to deliver a side-to-side anastomosis connector or an end-to-side type connector. Where a side-to-side connector is employed, either or both sides of the same may be deployed using the subject delivery device.

### FIELD OF THE INVENTION

The present invention is related to interconnecting conduits, especially blood vessels with anastomosis devices. More particularly, the present invention is related systems and methodology for delivering or implanting devices for interconnecting body conduits.

### BACKGROUND OF THE INVENTION

Millions of individuals are adversely affected by diseases of the cardiovascular system. In the context of the peripheral vascular and/or the cardiovascular system, atherosclerosis, a common vascular disease, can cause partial blockage or complete occlusion of an arterial vessel, resulting in restricted blood flow and therefore compromised perfusion to the tissue served by the blood flow. In the case of an occluded or partially occluded coronary vessel, for example, an area of the heart's myocardium would be compromised, which can lead to a myocardial infarction, or other ischemic heart syndrome such as congestive heart failure. In the case of peripheral vascular atherosclerotic disease, occluded vessels lead to ischemic syndromes such as threatened limbs, stroke and other morbidities. In many cases, such a blockage or restriction in the blood flow leading to the heart or peripheral vessels can be treated by a surgical procedure known as an artery bypass graft procedure. In the case of treating the heart, the procedure is known as a coronary artery bypass graft (CABG) procedure.

In any sort of bypass procedure, more-or-less normal blood flow is restored to an area either by directly attaching an available source vessel to the obstructed target artery or establishing a fluid connection between a source vessel and a target vessel with a live or synthetic graft. In either case, one or more anastomoses are formed between the lumen of interest, i.e., at least one connection is formed between one vessel and another or between a graft and adjoining vessels. A “side-to-side” anastomosis is formed by attaching the two conduits at locations (e.g., incisions/arteriotomies) along a side wall of each member. An “end-to-side” anastomosis is formed by attaching end of the first conduit to a location along a side wall of the second conduit.

Performing an anastomosis procedure can be very challenging. Particularly, a CABG procedure employing traditional surgical techniques involves cross-clamping the aorta, employing cardiopulmonary bypass support and tying multiple sutures. In addition, as cardiac surgery is moving to less invasive procedures, surgical access is being reduced—forcing surgeons to work in constantly smaller surgical fields.

CABG procedures are made more difficult due to the multiple characteristics that are unique to each anastomosis and to each patient. For example, the arteries' internal diameter dimensions are difficult to predict and the inside walls are often covered with deposits of stenotic plaque which creates the risk of dislodging plaque into the patient's blood stream during the anastomosis procedure. The resulting emboli in turn create a greater risk of stroke for the patient. The dislodgement of plaque is most likely to occur when the vessel wall undergoes trauma such as the puncturing, compression and tension exerted on the vessel by suturing and stapling. The vessel walls can also be friable and easy to tear, and are often covered with layers of fat and/or are deeply seated in the myocardium, adding to the difficulty of effectively and safely performing conventional anastomotic procedures. For at least these reasons, numerous anastomosis fittings/devices have been developed to simplify the procedure and/or reduce patient trauma.

In addressing these considerations, the assignee of the present invention produces devices offering advantages over other solutions. In particular, anastomotic connectors have been developed which avoid compression, tensioning and puncturing of the vessel tissue involved. Examples of such anastomotic connectors are disclosed in U.S. Pat. Nos. 6,165,185 and 6,251,116, and in U.S. Patent Application Publication No. US-2001-0044631-A1. These devices include at least one flexible member in the form of a sheet, membrane or flange that is adapted to conform to and seal with an inner surface or circumference of a vessel into which it is delivered. The flexible member is adapted to utilize only the internal vessel pressure, e.g., blood pressure, exerted thereon to form a substantially fluid-tight seal with the inner surface of the conduit whereby substances within the vessel are prevented from leaking from the artificial opening under normal physiological conditions. As such, these devices obviate the need to compress, puncture or place tension on the vessel tissue and reduce many of the risks associated with prior anastomotic and closure devices.

Another advantage of these flexible devices is that they can be made from materials which are biodegradable or bioresorbable, such as degradable hydrogels, polymers, protein cell matrices, plant or carbohydrate derivatives (sugars), and the like.

Of course, the referenced anastomosis devices can be employed in CABG procedures as well as a host of other applications. Such other applications include the creation of an arterialvenous fistula for the purpose of either creating a dialysis access site, or, as an alternative means of creating arterial revascularization by “arterializing” a vein through creation of a conduit past the occlusive disease. The latter is often employed in treating peripheral vascular disease but is used in coronary applications as well.

Whatever the application, aspects of the present invention address the manner in which vessels, natural body conduits or other conduits are anastomosed, often utilizing devices as referenced above. The invention is generally directed to delivery devices and methods associated in forming anastomoses as well as systems incorporating anastomosis devices or connectors suitable for use with the same.

### SUMMARY OF THE INVENTION

Delivery devices according to the present invention leverage the advantages presented by the referenced anastomotic device (though it may be used with others sharing certain functional similarities). In addition, delivery devices according to the present invention offer an easy to use and an uncumbersome manner of delivery, whereas many existing delivery devices are not so elegant in use or design. Further, a single configuration of a delivery device according to the present invention may be employed with a variety of configurations of anastomotic connectors, including both side-to-side and end-to-end devices. A single delivery device may also be suitable for delivery a number of differently-sized connectors. Moreover, delivery devices according to the present invention may be used for both proximal anastomosis applications, e.g., a graft vessel to the aorta, and distal anastomosis applications, e.g., a graft vessel to a native vessel at a location downstream of the stenotic lesion within the native vessel.

The invention includes hardware and/or methodology as described below in detail. Briefly, however, the invention involves a split-end confinement member, and plunger received within the confinement member. The confinement member is adapted to receive a connector, accommodating a transition section or tubular flow channel portion of the same, through an upper opening and able to deliver a seal portion through a lower opening. The confinement member may be further characterized as having a distal end that is angled backward from a lower distal tip. Optionally, a reinforcement member with an end that is split along its upper portion and being angled like the end of the confinement member may also be provided.

Methods according to the present invention involve delivery of an anastomotic connector using such hardware. In the methods, a distal end of the confinement tube and reinforcement tube distal end are introduced into an opening made in a vessel; next, a tubular flow channel portion of the anastomosis connector loaded into the delivery device is positioned adjacent a proximal end of the vessel opening; following such action, the reinforcement tube is withdrawn so its distal end clears a lower opening in the confinement tube; then, a plunger received within the confinement tube is advanced to urge a proximal portion of a seal of the anastomosis connector out of said confinement tube lower opening; finally, the confinement tube is withdrawn from the vessel, leaving a distal portion of the anastomosis seal in place within the vessel. The method may further comprise slightly rotating the confinement tube upward after withdrawing the reinforcement tube, but before withdrawing the confinement tube. Additional aspects of the method(s) and delivery device(s) may be provided as well.

### BRIEF DESCRIPTION OF THE FIGURES

To facilitate understanding of this disclosure, the same reference numerals have been used (where practical) to designate similar elements that are common to the Figures. Some such numbering has, however, been omitted for the sake of clarity.

FIG. 1A is a perspective view of a side-to-side anastomotic connector which is implantable using the delivery devices of the present invention; FIG. 1B is a top planar view of another side-to-side anastomotic connector which is implantable using the delivery devices of the present invention; and FIG. 1C is a perspective view of an end-to-side anastomotic connector which is implantable using the delivery devices of the present invention.

FIG. 2 is a transparent side view of components of a delivery device according to the present invention.

FIG. 3A is a side view of a confinement member according to the present invention; FIG. 3B is a perspective view of the component shown in FIG. 3A.

FIG. 4A is a side view of a reinforcement member according to the present invention; FIG. 4B is a perspective view of the component shown in FIG. 4A.

FIGS. 5A-5D are views showing preparation of a delivery device with a connector according to the present invention, of which FIGS. 5A-5C are perspective views and FIG. 5D is a close-up view of a portion of FIG. 5C taken from the side.

FIGS. 6A-6E are perspective views of delivery of an anastomotic connector at various stages according to the present invention.

### DETAILED DESCRIPTION OF THE INVENTION

In describing the invention in greater detail than provided in the Summary and as informed by the Background above, suitable hardware for use in the invention is first described. Namely, exemplary anastomotic connectors are described as well as the subject delivery devices. This discussion is followed by description of method of using the same according to the present invention. Finally, a review of the kits of the present invention which include the subject delivery systems and devices for performing the subject methods is provided.

In the following description, the present invention as used in anastomotic applications will be described in the context of joining two vessels wherein at least one of the vessels is the target vessel to be bypassed such as a coronary or peripheral vessel. The other vessel is a graft vessel which may be pedicled or segmented from its native location. However, such exemplary application is not intended to be limiting and those skilled in the art will appreciate that the subject devices, systems and methods are useful for the joining of other types of conduits and structures and may be used to join any numbers of vessels or other conduits and structures, i.e., may be used to join greater than two vessels or other conduits and structures.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

Anastomotic Connectors

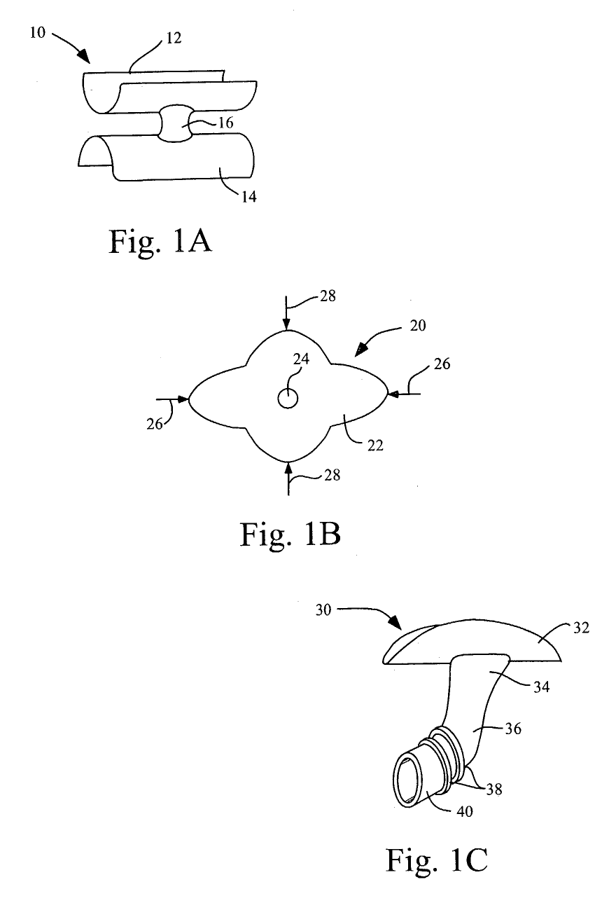

FIGS. 1A-1C illustrate various embodiments of the anastomotic connectors generally described above which are suitable for use with the present invention. As alluded to above, such devices are described in detail in U.S. Pat. Nos. 6,165,185, and 6,251,116, U.S. Patent Application Publication No. US-2001-0044631-Al, and U.S. Patent Application Attorney Docket No. VASC-009 to Akin, et al, filed on even date herewith.

While the subject invention is especially useful for delivering the anastomotic connectors disclosed in these documents, it will be obvious to those skilled in the art that the subject devices and methods herein described may be employed with other anastomotic connectors. As such, reference to specific embodiments of anastomotic connectors is solely for purposes of describing the subject invention and is not in any way intended to limit the scope or the function of the subject invention. While reference can be made to the above-referenced patents for a detailed description of anastomotic connectors usable with the present invention, a brief description is herein provided for purposes of convenience.

Each anastomotic device comprises at least one flexible seal member in the form of a sheet, membrane or flange. The devices configured for forming a side-to-side anastomotic connection include a second flexible seal wherein a flow opening or channel resides between the members such that they are in fluid communication. Those connectors configured for forming an end-to-end anastomotic connection have a second member having a tubular configuration wherein the lumen of the tubular member extends from a flow opening in the flexible member.

In either configuration, the flexible members are adapted to conform to and seal with an inner surface or circumference of a vessel into which it is delivered. Furthermore, each such flexible member is adapted to utilize the internal vessel pressure exerted thereon to form a substantially fluid-tight seal with the inner surface of the conduit whereby substances within the vessel are prevented from leaking from the artificial opening under normal physiological conditions. More particularly, the flexible seal member has first and second surfaces. The first or lumen-facing surface is adapted to utilize the internal conduit pressure exerted thereon to form a substantially fluid-tight seal between the second or vessel-contacting surface and an inner wall or circumference of the vessel. Thus, upon deployment of the flexible member into a vessel, the member conforms to the interior walls of the vessel to provide a sealing contact and sufficient physical stability to the device to prevent displacement from the vessel. Moreover, the substantially fluid-tight seal is formed without compressing, tensioning or puncturing the vessel wall.

The seal members are constrictable or constrainable (such as by bending or folding) to a size sufficient to fit through the artificial opening and are expandable to be securely and permanently self-retained within the vessel upon implantation. The flexible members comprise relatively thin walls, thus minimally interfering with fluid flow within the interconnected vessels. The intravascular pressure against the underside of the flexible member secures the member against the inside vessel wall thereby preventing leakage from the anastomosis site. Additionally, the configuration of the flexible seal members is such that it provides an element of passive force when deployed within the vessel so as to pull the two vessels together for better sealing and healing of the vessel walls.

In certain embodiments the seal flanges have constant diameters about their circumference (e.g., circular) or the same length and width dimensions (e.g., square). In other embodiments, the flanges have varying diameters (e.g., elliptical, oval) or lengths and widths (e.g., rectangular), wherein the flanges have a major axis, i.e., a longer axis, and a minor axis, i.e., a shorter axis. In any embodiment, the flexible membranes are sufficiently flexible and compliant to be folded about any axis defined by the membranes, as well as to be folded about an axis which is defined by the flow channel, which may be substantially perpendicular to the surface of the flange or at angle. Such flexibility facilitates implantation of the anastomotic connectors with the devices of the present invention.

Upon release of the seal flanges/membranes from a constricted or folded condition, each subsequently conforms to the interior walls of a conduit to provide a sealing contact along the contact surface of the membrane. Once deployed within the conduits, the sealing contact and stiffness properties of the flanges provide sufficient physical stability to the device to prevent displacement from the respective vessels.

The flexible seal flanges may have a variety of different configurations, shape(s), thickness(es), surface areas, lengths and widths (or diameters). Useful configurations include, but are not limited to, partial cylinders or generally planar configurations having circular elliptical, stared, petaled or rectangular shapes, or combinations of these configurations.

Each flange or membrane seal member includes an opening through its thickness which provides a pathway through which fluid can be transported between anastomosed conduits. More specifically, the flow opening provides a location of permanent connection between the two members of the anastomosis device, whether a side-to-side or an end-to-side device, and thus, establishes fluid communication between the vessels connected by the implanted device.

Generally, the size and shape of the flexible members are dependent on the size (i.e., the circumference or diameter) and shape of the bodily lumen into which it is to be used. For example, larger segments may be preferable when performing a proximal anastomosis to an aorta, or when anastomosing peripheral (e.g., in the leg) or abdominal vessels while smaller segments are more appropriate for coronary arteries and veins. Also, the length or width (or diameter) dimensions or both, may be dictated by the length of the incision or arteriotomy within the lumen or vessel into which the segment is to be placed.

The anastomotic connectors may be made of biodegradable or bioresorbable materials or non-resorbable materials. Suitable bioresorabable materials include but are not limited to degradable hydrogels, polymers such as lactides/glycolides or PHAs; protein cell matrices, plant, carbohydrate derivatives (sugars), and the like. Suitable non-resorbable materials include but are not limited to polymers and elastomers such as silicones, fluoropolymers, polyolephins or polyurethanes might also be used. In addition, the anastomotic connectors may be fabricated from composites of two or more different types of materials, etc, e.g., the device may be fabricated from a blood impermeable membrane attached to a structural article or scaffold.

In addition to being adequately biocompatible, the material(s) have appropriate mechanical properties for facilitating insertion, retention and sealing of the members within the vessels. Additionally, the anastomotic connectors may be made of any suitable autologous, allo- and xeno-graft biomaterials.

Referring now to the figures, specific embodiments of anastomotic connectors are illustrated that are usable with the present invention. The side-to-side anastomotic connector 10 of FIG. 1A includes both a first portion or flexible member, membrane or flange type seal 12 and a second portion or flexible member, membrane or flange type seal 14 connected by a tubular flow channel 16 which extends between the two flanges to provide fluid communication between the vessels into which flanges 12 and 14 are inserted. In this embodiment, each seal 12, 14 has a rectangular contact surface which, when in a constricted condition along the longitudinal axis of the flange, has a semi-cylindrical configuration.

FIG. 1B illustrates a top view of another side-to-side anastomotic connector 20 having a first portion or flange/seal 22 having a petal configuration and a flow opening 24 and a second portion or flange/seal and associated flow opening (neither of which are shown) which correspond in size and shape to flange 22 and opening 24, respectively. Between the flow openings extends a tubular portion or flow channel (not shown) similar to that shown in FIG. 1A.

As mentioned above, the side-to-side or end-to-end distances of flange 22, designated by arrows 26 and 28, may be the same or differ from each other. In certain embodiments, the flanges may have a major axis, such as defined by arrows 26, and a minor axis, such as defined by arrows 28. The seals are bendable or foldable about either axis, and thus, device 20 may be implanted in either folded configuration as required by the surgical application. The petal-shaped device 20 is particularly useful with the delivery devices of the present invention as its flanges are easily foldable about the axis defined by its flow channel.

FIG. 1C illustrates an end-to-side anastomotic connector 30 having a first portion or membrane or flange seal member 32 having an oval shape and a second portion or tubular member 36 joined together at a flow opening, defined externally by juncture 34, analogous to that found in the side-to-side devices described above. The seal member 32 of the end-to-side device has the same or similar properties and advantages as described above with respect to the flange/seal members of the side-to-side device. Flange 32 is shown as a partial cylinder having an elliptically shaped contact surface.

Tubular flow channel 36 may be normal to, or positioned at an angle relative to, the surface of flange member 32. Tubular member 36 is designed to fit inside of the transected end of a graft that is to be joined to the side of a host vessel. The length of tubular member 36 typically ranges from about 10 to 20 mm. The outer diameter of tubular member 36 has a dimension that approximates the inner diameter of the synthetic graft or graft vessel to be attached, and therefore is typically in the range from about 2 to 6 mm, and more typically from about 3 to 5 mm. Optionally, tubular member 36 has a vessel securement means 38 for further securing tubular member 36 within a graft vessel. Here, vessel securement means is in the form of two parallel rings surrounding the circumference of tubular member and appropriately positioned vis-à-vis the host vessel, another component of the securement means (not shown) such as a cuff or ring may be temporarily or permanently positioned about the graft vessel and within the spacing formed by the parallel rings.

Delivery Devices

The above-referenced anastomosis devices are advantageously compressed in order to be delivered through a small hole or incision in the target vessels. Yet, the physical properties of the materials used in such devices may require that the devices are packaged/stored in an unconstrained or unstressed configuration, and then loaded into a delivery system in the operating theater. Otherwise, permanent deformation or plastic deformation may occur. Discussion of device loading and deployment will follow a description of the subject delivery devices.

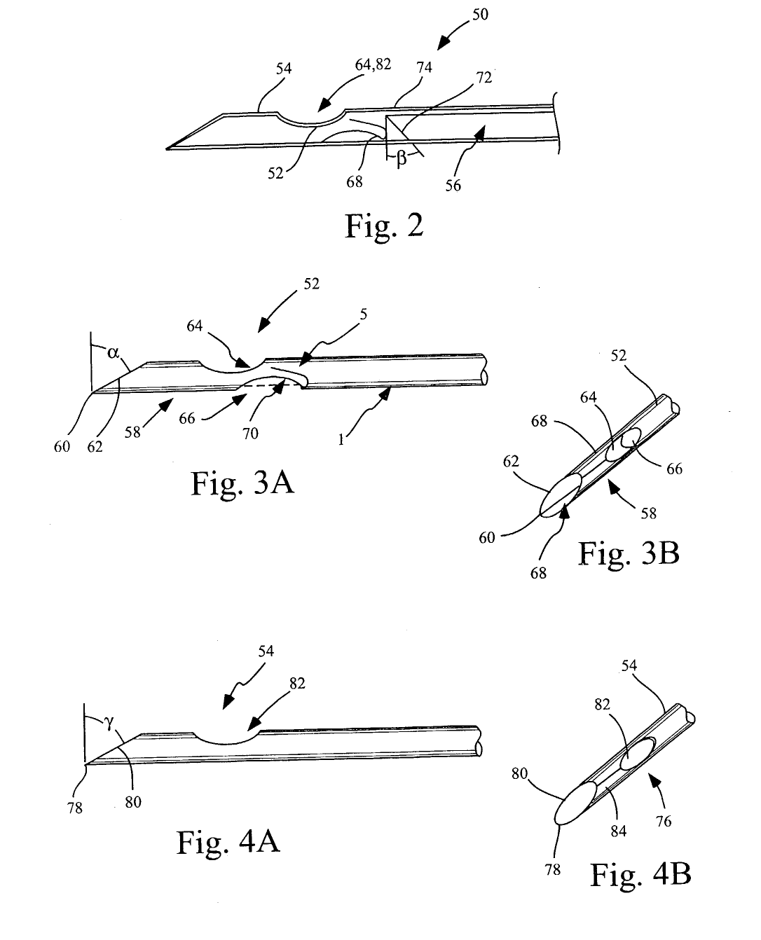

Regarding such devices, an exemplary system 50 according to the present invention is shown with each of its optional constituent components in FIG. 2. These components include an inner or confinement/capture tube 52 and an outer or reinforcement tube or sheath 54 and a pusher or plunger mechanism 56. At minimum, the system for anastomosis device delivery will include at least the capture tube 52 and pusher 56 or its equivalent. In instances where the reinforcement member is omitted, however, it may be desired to integrally reinforce the structure of confinement member so it can function as intended.

FIG. 3A presents a side view of confinement member 52; FIG. 3B is a perspective view of a distal end 58 of the same. The distal end is preferably angled backward from a lower distal tip 60. The angle α formed between a vertical and the tube rim or edge 62 is preferably between about 20 and about 70 degrees.

Regardless, confinement member 52 includes upper and lower openings, 64 and 66 respectively. As may be observed, at least a portion of upper opening 64 is positioned distal to (in front or ahead of) lower opening 66. The confinement tube is split along upper and lower portions distal of the upper and lower openings. The skives or slits along confinement member 52 tubular body provide substantially independent distal side sections 68.

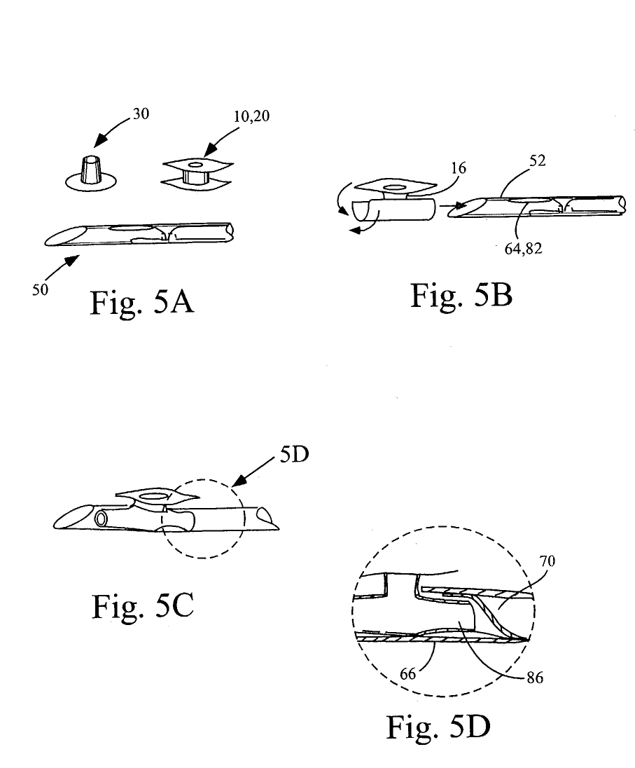

In addition, a flap or pusher 70 may be provided in connection with confinement member 52. The flap may be formed by a section of material cut-out adjacent lower opening 66. Once the tube is cut to leave a flap, the material can be folded upwards into the lumen of the tube to act as a lever mechanism to assist deployment of an anastomosis connector. As shown in FIG. 5D, the flap may be provided by a separate piece of material. Still, some form of a unitary construction may be preferred, though there are many options for producing flap 70.

The flap portion of the delivery device is typically urged forward and/or downward by a distal surface 72 of plunger 56. The end of the plunger may be angled or configured otherwise. Preferably, it is angled backward from an upper distal tip 74 when setup as shown in FIG. 2 as desired. The angle α formed between a vertical and the distal surface may be between about 20 and about 70 degrees, as may be convenient in view of flap configuration or construction.

Regarding outer tube 54, which serves as a reinforcement member to confinement member 52 when employed, FIG. 4A provides a side view of the device in isolation. FIG. 4B is a perspective view of a distal end 76 of the same. As with the confinement member, the distal end is shown angled backward from a lower distal tip 78. The angle γ formed between a vertical and the tube rim or edge 80 preferably matches angle α, though mismatched angles may be suitable in certain circumstances.

An upper opening 82 is provided along reinforcement member 54. At least a portion of this opening, preferably the entire opening, is positioned to align with the upper opening 64 of confinement member 52, substantially as shown in FIG. 2.

Reinforcement tube 54 is split along an upper portion distal to the upper opening. The skive or slit along its tubular body provides a split-tube section 84 that may be opened, but may offer greater structural integrity than independent distal side sections 66 of confinement tube 52.

Plunger 56 may be comprised of a hollow or solid rod slideably disposed within confinement tube 52. The plunger has a distal end 72 which may be angled toward the flap 70. Preferably, it is angled as shown in FIG. 2. The angle β formed between a vertical and the distal surface may be between about 20 and about 70 degrees. Furthermore, the plunger may also incorporate an active mechanism at the distal end to actuate the flap of the confinement tube.

Both tubes preferably comprise thin walled (between about 0.004 and 0.020 inch wall thickness) plastic, e.g., polyethylene, polypropylene, PVC, urethane, PEBAX or similar polymer. However, they are preferably made from a fluoropolymer such as TFE or FEP. The plunger may be of any conventional construction, though use of like material may be desired.

Material selection, and the sizing or particular spacing of components is within the level of one with skill in the art. Likewise, while simple tubular and/or rod-like members may be employed in the present invention, more complex structures including handle pieces, especially ergonomic handles, at proximal portions of the respective elements may be employed.

Methods

Irrespective of such potential variation noted and yet other variation as may be applied to the disclosed structural aspects of the present invention, certain methodology will be employed in use. This methodology typically begins as shown in FIG. 5A in taking a kit including at least one connector 10, 20 or 30 and a delivery device 50 or bringing together an anastomosis connector that is sized for a given anastomosis procedure and a delivery device suitably sized to work with the same. As illustrated in FIG. 5B, a/the connector flange is compressed for insertion into the delivery device. A folding or rolling-type approach is depicted in which opposite “wings” of the flange compressed along mayor axis 26 (the minor axis 26 portions of flange 14, 22 are inwardly deformed. In connection with a preferred connector device, the approach of the invention enables the flange(s) to be folded or compressed tightly only in their direction of curvature, which minimizes the stress on the polymer.

However the anastomosis connector is prepared for loading, it is inserted into the delivery device/system as depicted in FIG. 5B until set in place as shown in FIG. 5C to form an anastomosis system, ready for use/operation. To do so, the tubular portion 16, 36 of the anastomosis device slides past the slit in the confinement tube (and reinforcement tube 54—when one is used) until it is located within opening 64 (and opening 82 of the reinforcement tube when one is used). Such action causes side sections 70 (and split tube section 82—when reinforcement tube 54 is used) to flex open to allow the anastomosis connector pass to a state ready for deployment. In preparing a device as such, tubular/stoma section 16, 36 (of the respective connectors 10 and 30, for example) is compressed as it is advanced through the distal slits in the tube(s). The connector stoma section 16, 36 then resides in a less constrained, or even an uncompressed, position in the top opening(s) 64, 82 of tubes 52, 54, respectively.

As shown in the transparent or cross-sectional detailed view of the region indicated in FIG. 5C, FIG. 5D pictures a proximal flange portion 86 tucked under flap surface 70 opposite opening 66. Once the plunger is inserted (if it has not already been inserted), the delivery system 50 is ready for action.

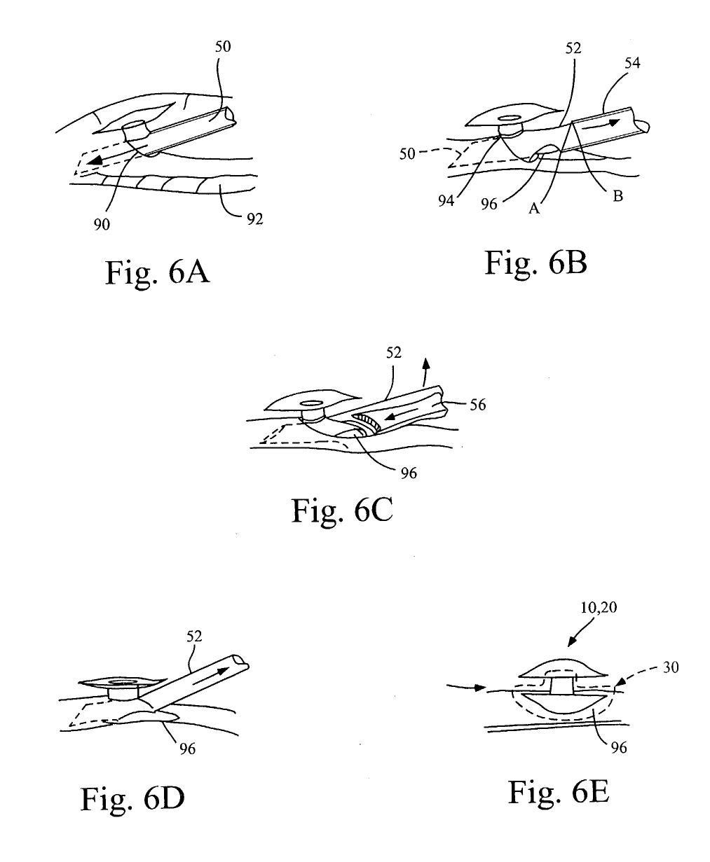

FIGS. 6A-6E depict the activity carried out in performing at least part of an anastomosis procedure with the delivery system. After an opening 90 has been formed in the wall of a target vessel 92 (usually a small incision produced by a scalpel as in the case of an arteriotomy in a coronary artery anastomosis procedure), the distal end of delivery device 50 is inserted therein.

The angled or beveled end of the device as provided by the angled end of both confinement member 52 and reinforcement member 54, reinforcement member 54 alone (provided the end of the confinement member is set proximal to edge 62), or confinement member 52 (when used alone), offers a lead-in to assist vessel entry. Once insertion into the target vessel has been achieved, the delivery device 50 and anastomosis connector are advanced until the tubular portion of the connector abuts the forward limit 94 of incision 90.

Next reinforcement member 54 is at least partially withdrawn. It is taken back or translated proximally at least to a point where opening 66 is cleared (at least to the point at which it is aligned with flange proximal end 86, point “A”). In abundance of caution, member 54 may be retracted over member 52 to a point “B” some distance from point “A.” However far back member 54 is withdrawn, to do so, the split end portions 84 open partially to pass by the tubular connection portion 16, 36 of the anastomosis device.

Following retraction of member 54, pusher 56 is advanced to urge flap 70 forward to eject the anastomosis proximal flange or seal portion/heel 96 out of opening 66. Such action urges, protrudes or “pops” the heel of the anastomosis connector out as desired. Prior to advancing the plunger, however, it may be desired to slightly tilt or rotate at least the confinement member 52 and plunger 56 upward. The rotation may be “slight” (i.e., between about 10 and about 30 degrees) or more extreme (i.e., between about 45 and 90.)

Doing so may provide an improved angle or route for flange exit. In any case, such activity is depicted in FIG. 6C. The pusher is shown as it folds the flange heel portion 96 downward toward the vessel lumen, allowing it to clear the back edge of the incision as it exists the “trap door” opening 66.

Once the anastomosis device is emplaced, the confinement or introducer tube 52 is withdrawn as shown in FIG. 6D. In retracting this portion of the delivery device, both split side sections 68 open to pass the connector tubular portion. The result of the completed anastomosis procedure is shown in FIG. 6E.

In the case where a two-sided connector is provided as shown, the same steps may be taken to place the second flange in-situ. In instances where a single-sided connector is provided, a graft vessel may be manually attached to the tubular pathway portion of the connector, before or after the flange is placed.

Where no reinforcement member 54 is provided in the system, the anastomosis procedure will be accomplished by omitting the action shown in FIG. 6B. In this case, the anastomosis connector and confinement sheath 52 will be all that is introduced into vessel 92 in the step shown in FIG. 6A. However, it is noted that providing a reinforcement member offers great benefit in strength and stability to the system. Certain configurations may not be able to operate without the outer sheath and the additional axial stiffness it offers.

Other variation specifically contemplated for the invention involves providing a pusher in the form of an active mechanism. Examples of such mechanisms may incorporate a spring loaded lever-arm actuated or pre-stressed finger section to push the flange downward into the target vessel mechanism upon activation.

### EXAMPLE

Depending on the size or nature of an anastomosis connector to be used in connection with the delivery device, the size and the relative spacing of delivery device features may vary. In a variation adapted to deploy connectors to a coronary artery, openings 64 and 82 were set approximately 1 cm back along the top surface from the top of rim 62 and 80, respectively. Opening 66 was located along the bottom surface of member 54 starting generally from the midline of the upper opening 64 and proceeding proximally for about 1 cm.

Such an implantation tool was tested in thin walled silicone tubes, serving as artificial vessels, in ex-vivo porcine vessels and in-vivo porcine vessels with success in each case. The in-vivo testing involved two porcine models. In the first, a medium sized, multi-lobe “clover” flanged STS device as shown in FIG. 1B was used in the proximal anastomosis site of a carotid bypass. The graft vessel was a segment of excised femoral artery. Deployment success rates of up to 90% were achieved with a prototype device. Complete flange deployment was easily accomplished, with a delivery time (per flange) of less than 1 minute.

In the second porcine study, an anastomosis of the femoral artery was evaluated. Here, a small “clover” device was utilized and successfully deployed multiple times, as above, even using a delivery tool more appropriately sized for the larger device. In further practice of the invention, deployment tools with sizing tailored to the particular size of the anastomosis connector may generally be preferred. Yet, it has been demonstrated that a single delivery device offers flexibility to deploy connectors of different sizes.

Though the invention has been described in reference to certain examples, optionally incorporating various features, the invention is not to be limited to the set-ups described. The invention is not limited to the uses noted or by way of the exemplary description provided herein. It is to be understood that the breadth of the present invention is to be limited only by the literal or equitable scope of the following claims. That being said,

We claim:

1. An anastomosis system for connecting a target vessel with a graft vessel, said system comprising:
a delivery device comprising a confinement tube having a proximal end and a distal end, said distal end angled backward from a lower distal tip, said confinement tube having an upper and a lower opening, at least a portion of said upper opening positioned distal to said lower opening, said confinement tube being split along upper and lower portions distal of said upper and lower openings, thereby providing independent distal side sections, said delivery device further comprising a plunger adapted to be received within said confinement tube.

2. The system of claim 1, wherein said plunger has a distal end angled backward between about 20 and about 70 from a lower distal tip.

3. The system of claim 1, wherein said tube further comprises a flap proximal to said lower opening wherein said flap and said plunger are adapted so forward movement of said plunger urges said flap downward.

4. The system of claim 3, wherein said flap is formed by a loop cut from said tube.

5. The system of claim 3, wherein a distal end of said plunger includes an active mechanism to actuate said flap.

6. The system of claim 1, wherein said distal end of said confinement tube is angled between about 20° and about 70°.

7. The system of claim 1, wherein said delivery device further comprises a reinforcement tube having a proximal end and a distal end, said distal end angled backward, said reinforcement tube having an upper opening and being split along an upper portion distal to said upper opening, wherein said reinforcement tube is adapted to closely fit over said confinement tube, with said upper openings of each tube being aligned.

8. The system of claim 7, wherein said distal end of said reinforcement tube is angled between about 20° and about 70°.

9. The system of claim 7, wherein said distal ends have a matched angle.

10. The system of claim 1, further comprising an anastomosis connector comprising a flexible seal portion adapted to fit within said confinement tube upon compression, and a tubular flow channel portion adapted to be received by said confinement tube upper opening, wherein said confinement tube lower opening is positioned to be below a proximal portion of said seal portion when said anastomosis device is in place within said confinement tuber.

11. The system of claim 10, further comprising a reinforcement tube as described in claim 6.

12. The system of claim 10, wherein said anastomosis device further comprises a graft attachment tube extending from said tubular flow channel portion.

13. The system of claim 10, wherein said anastomosis device further comprises another flexible seal portion at said tubular flow channel portion.

14. A method of delivering an anastomosis device, said method comprising:
providing a system as describe in claim 10,
advancing said confinement tube distal end and said reinforcement tube distal end into an opening made in a vessel,
positioning said tubular flow channel portion adjacent a proximal end of the opening in the vessel,
withdrawing said reinforcement tube so its distal end clears said lower opening of said confinement tube,
advancing said plunger to urge a proximal portion of said anastomosis seal out of said confinement tube lower opening, and
withdrawing said confinement tube from said vessel, leaving a distal portion of said anastomosis seal in place within the vessel.

15. The method of claim 14, further comprising rotating at least said confinement tube upward by up to about 15° after withdrawing said reinforcement tube and before withdrawing said confinement tube.

16. The method of claim 14, wherein said confinement tube further comprises a flap and said plunger engages said flap upon advancing said plunger.

17. The method of claim 14, further comprising attaching a graft to said anastomosis device.

18. The method of claim 14, wherein an end-to-side anastomosis is produced.

19. The method of claim 18, further comprising maintaining said anastomosis at least partially by the blood pressure of said vessel and said graft.

20. The method of claim 14, wherein a side-to-side anastomosis is produced by repeating the acts of claim 14 for another flexible seal portion of said anastomosis device.

21. The method of claim 20, further comprising maintaining said anastomosis at least partially by the blood pressure of said vessel and said graft.

22. An anastomosis system made by providing an anastomosis delivery system as described in claim 10, loading said anastomosis device into said confinement tube by inwardly deforming said seal portion and passing said tubular flow channel portion through said upper opening of said confinement tube, and advancing said reinforcement tube over said confinement tube, aligning said tubular flow channel portion with said upper opening of said reinforcement tube.

23. The anastomosis system of claim 22, further made by advancing said plunger into said confinement tube so said distal end of said plunger is adjacent said a proximal end of said anastomosis device seal.

24. The anastomosis system of claim 22, wherein said confinement tube further comprises a flap and the system is further made by advancing said plunger into said confinement tube so said distal end of said plunger is adjacent said flap and said flap is adjacent a proximal end of said anastomosis device seal.
